(19) United States
(12) Patent Application Publication
(10) Pub. No.: US 2026/0110633 A1
(43) Pub. Date: Apr. 23, 2026

(54) FLUID SENSING DEVICE AND CONTROL SYSTEM

(71) Applicant: Brolis Sensor Technology, UAB, Vilnius (LT)

(72) Inventors: Tadas Buciunas, Vilnius (LT); Benediktas Bilinskas, Vilnius (LT); Augustas Baltrenas, Vilnius (LT); Kristijonas Vizbaras, Vilnius (LT); Augustinas Vizbaras, Vilnius (LT)

(21) Appl. No.: 19/118,134

(22) PCT Filed: Oct. 6, 2023
(86) PCT No.: PCT/IB2023/060062

§ 371 Date: Apr. 3, 2025

Related U.S. Application Data
(60) Provisional application No. 63/414,065, filed on Oct. 7, 2022.

(51) Int. Cl. G01N 21/39; G01N 21/85; G01N 33/04

(52) U.S. Cl. CPC G01N 21/39; G01N 21/85; G01N 33/04

(57) ABSTRACT

A system for determining characteristics of milk or other fluid flowing through an inline sensor system includes a laser engine comprising that emits a laser radiation through the milk as the milk flows through the sensing system, a laser detector that receives the laser after the laser has passed through the milk and generates corresponding laser-readings, one or more processors, and computer memory storing computer-readable instructions. The instructions cause the processors to receive, from the laser detector, laser-readings from the laser detector, identify spectral data reflective of physical properties of the fluid as the fluid flows through the sensing system from the laser-readings, and determine one or more fluid-measures for corresponding one or more constituents of the fluid using the spectra-data and reference-data defining one or more reference spectra for each possible constituent of the fluid.

### CROSS-REFERENCE TO RELATED APPLICATION

This application claims the benefit of U.S. Provisional Application No. 63/414,065 filed Oct. 7, 2022, the disclosure of which is incorporated herein in its entirety.

### TECHNICAL FIELD

This document describes automated control and sensing using a laser.

### BACKGROUND

A tunable laser is a laser with a wavelength of operation that can be altered in a controlled manner. Most or all laser gain media allow small shifts in output wavelength, and some such laser gain media also allow continuous tuning over a more significant wavelength range. Gas, liquid, and solid-state lasers exist. Some examples include excimer lasers, gas lasers, dye lasers, solid-state lasers, semiconductor crystal and diode lasers, and free electron lasers.

Industrial controls systems (ICS) include electronic control systems and associated instrumentation that can be used for industrial process control. Control systems can range in sizes that include a few modular panel-mounted controllers to large interconnected and interactive distributed control systems. Some control systems are implemented by supervisory control and data acquisition (SCADA) and/or programmable logic controllers (PLC).

Dairy farms involve lactating mammals such as cows, goats, sheep, etc., for milk production, which in turn is used for a range of other dairy products such as fluid milk, anhydrous milk fat, whole milk powder, lactose, cheese, butter, yogurt, cream, kefir, etc. Milk and milk products are an integral and essential part of the global food sector, and dairy farms are responsible for the raw material production. Production efficiency, sustainability and, in the end, profitability depends on a number of factors but those of key importance are efficient herd health and herd selection management.

### SUMMARY

In-line electro-optical sensor for real-time composition analysis may be implemented in different industrial, agricultural, and biomedical environments where a flowing through liquid phase, solid phase or gas phase substance is presented. In particular, in-line real-time monitoring of milk composition in the milking line for individual animal is presented, combined with a method of utilizing the measured composition data to aggregate and construct both short-term and long-term data trends and build overall process optimization models to bolster farm efficiency in terms of output, minimal animal down time and controlled herd selection. Some embodiments of the invention use a combined electro-optical in-line sensor within a milking line to monitor and aggregate real-time or nearly real-time milk composition data for every individual milking process for every animal of the herd. Aggregated data over time can be used by control systems to proactively provide early warning indication of the animal health, in such way providing opportunity for timely treatment, potentially at a fraction of a cost both in medication as well as animal down time. Post-treatment animal output and milk composition monitoring for data trending may provide information of treatment efficacy, which, in turn, could help farmer establish most efficient way of treating one or another animal health issue as well as compare different available medication options. In addition, individual animal monitoring within the herd provides direct data for herd selection based on a particular desired trait such as high protein, and/or high fat content, disease resistance etc., leading to a controlled process and improved efficiency as well as profitability of the business.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a sensing system for sensing physical properties of a fluid. The system includes a laser engine that may be configured to emit spectrally tunable laser radiation through the fluid as the fluid flows through the sensing system. The sensing system also includes a laser detector configured to receive the laser radiation after the laser radiation has passed through the fluid and to generate corresponding laser-readings. The system also includes one or more processors. The system also includes computer memory storing computer-readable instructions that, when executed by the one or more processors, cause the one or more processors to perform operations that may include: receiving, from the laser detector, laser-readings from the laser detector; identifying, from the laser-readings, spectral-data reflective of physical properties of the fluid as the fluid flows through the sensing system; and determining one or more fluid-measures for corresponding one or more constituents of the fluid using the spectra-data and reference-data defining one or more reference spectra for each possible constituent of the fluid. Other embodiments of this aspect include corresponding computer systems, apparatus, and computer programs recorded on one or more computer storage devices, each configured to perform the actions of the methods.

Implementations may include one or more of the following features. The system where the system further may include a housing containing the laser engine, the laser detector, and one or more processors, and the computer memory. The system further may include a network interface, and where the operations further may include transmitting, through the network interface, the one or more fluid-measures. The system further may include: a housing containing the laser engine and the laser detector; and one or more computing devices may include the one or more processors and the computer memory. The system further may include a fluid channel through which the fluid flows between the laser engine and the laser detector, and where the laser engine and laser detector are fixedly held at least partly in the fluid channel such that a portion of the fluid flows between the laser engine and the laser detector. The laser engine and laser detector are fixedly held apart at a distance of less than 10 mm. The laser engine is coupled to a photon-permeable sheath configured to prevent contact of the laser engine by the fluid. The system further may include: a contact sensor fixedly held at least partly in the fluid channel; an optical emitter fixedly held at least partly in the fluid channel; and a color sensor fixedly held at least partly in the fluid channel. The system further may include a contact sensor configured to: contact the fluid as the fluid flows through the sensing system; sense one or more contact-phenomenon of the fluid to create corresponding contact-readings; and where determining one or more fluid-measures further may include using the contact-readings. The system further may include: an optical emitter configured to emit a non-coherent light; and a color sensor configured to receive the non-coherent light after the non-coherent light has passed through the fluid and to generate corresponding non-coherent-readings; and where determining one or more fluid-measures further may include using the non-coherent-readings. The system further may include an automated valve configured to selectively direct a flow of the fluid to a plurality of output channels; and where the operations may include actuating the automated valve based on at least one of the fluid-measures. Actuating the automated valve may include engaging the automated valve to direct the fluid to a waste tank responsive to determining a fluid-measure for a contaminant is greater than a threshold value. Actuating the automated valve based on at least one of the fluid-measures may include actuating the automated valve to direct the fluid to the waste tank before contaminated fluid reaches the automated valve. The fluid is raw milk harvested from livestock. The laser engine may include an III-V semiconductor based laser. The III-V semiconductor based laser is configured to emit the light through an optical interface, and where the laser detector is positioned opposite the optical interface. The III-V semiconductor based laser is a tunable laser. The III-V semiconductor based laser is configured to emit the light through the optical interface over a spectrum of wavelengths. The laser engine may include an III-V/IV semiconductor based laser. The III-V/IV semiconductor based laser is configured to emit the light through an optical interface, and where the laser detector is positioned opposite the optical interface. The III-V/IV semiconductor based laser is a tunable laser. The III-V/IV semiconductor based laser is configured to emit the light through the optical interface over a spectrum of wavelengths. The optical interface is positioned in a flow path of the fluid flowing through the sensing system. The optical interface may include a tube extending from the laser engine into a channel through which the fluid flows through the sensing system, the tube extending toward the laser detector, where a distance from a distal end of the glass tube to a surface of the laser detector is between 0.5 mm and 10 mm, the tube may include an optically transparent portion. The fluid-measures are each measures of constituents at a particular time, the operations further may include: aggregating the fluid-measures of a single milking session into an aggregate that reflects changes to the fluid-measure through the milking session. The operations further may include: storing the fluid-measures of a single milking session indexed by a unique identifier for the single milking session for an individual animal. The operations further may include: storing the fluid-measures of a single milking session with other fluid-measures of other milking sessions to generate historic data for a particular animal. The operations further may include: generating herd-wise metrics for a plurality of animals using historic data for each animal in a herd. The operations further may include: matching the herd-wise metrics with third party data to at least one of the group may include 1) herd health data, 2) herd selection data, and 3) herd management data. Generation of individual or herd-wise metrics includes the use of the third party data and historic data for each animal in a herd. Physical properties of the fluid include fluid composition, electrical properties, temperature, flow, color, quantitate constituent concentration levels, absence or presence of one or more constituents. Implementations of the described techniques may include hardware, a method or process, or computer software on a computer-accessible medium.

Other features, aspects and potential advantages will be apparent from the accompanying description and figures.

### DESCRIPTION OF DRAWINGS

FIGS. 1A and 1B show example systems for harvesting milk from livestock using electro-optical inline sensors including tunable laser spectral sensors.

FIGS. 2A-2B show example devices for sensing phenomena of fluids.

FIGS. 3A and 3B shows example devices for sensing phenomena of fluids.

FIG. 4 shows an example fluid channel.

FIG. 5 shows hardware architecture that can be used.

FIG. 6 shows processing to generate unique animal milk composition records.

FIG. 7 shows a model.

FIG. 8A-8D show generation of data from raw sensor data.

FIG. 9 is a swimlane diagram of an example process for sensing fluid.

FIG. 10 shows data illustrating absorption spectra of milk for a variety of transmission path distances measured between the optical interface and laser detector.

FIG. 11 shows example data usable in sensing fluids and controlling automated devices.

FIG. 12 shows a swimlane diagram of an example process for sensing fluid usable in a milk-harvesting system.

FIG. 13 shows a flowchart of an example process for diverting contaminated fluids away from a holding tank and into a waste tank.

FIG. 14 is a schematic diagram that shows an example of a computing device and a mobile computing device.

Like reference symbols in the various drawings indicate like elements

### DETAILED DESCRIPTION

An inline sensor can use one or more sensors to detect one or more properties of a fluid as the fluid passes through the inline sensor. For example, an electro-optical sensor of the inline sensor can be used to sense properties of raw milk as the raw milk is being harvested from a cow or other livestock. Based on the sensing performed by the inline sensor, one or more automated devices can be actuated by computer control. For example, an automated valve in the fluid line can direct the flow of the raw milk to a waste tank if a contaminant (e.g., blood, high somatic cell count, etc.) is identified in the milk. The information from the inline sensors can also be used to perform composition analysis to determine other properties of the milk. The determined properties can be used to make decisions about on-going milk collection (e.g., high fat-content milk directed to a particular tank, identification of incorrectly positioned milking apparatus, etc.) and can be aggregated with other historical and third-party information to identify trends related to animals within a herd and output of a particular farm. Additionally, the aggregated data can provide information about the health and production of the herd that can be used to make decisions about herd health, selection, management, and care.

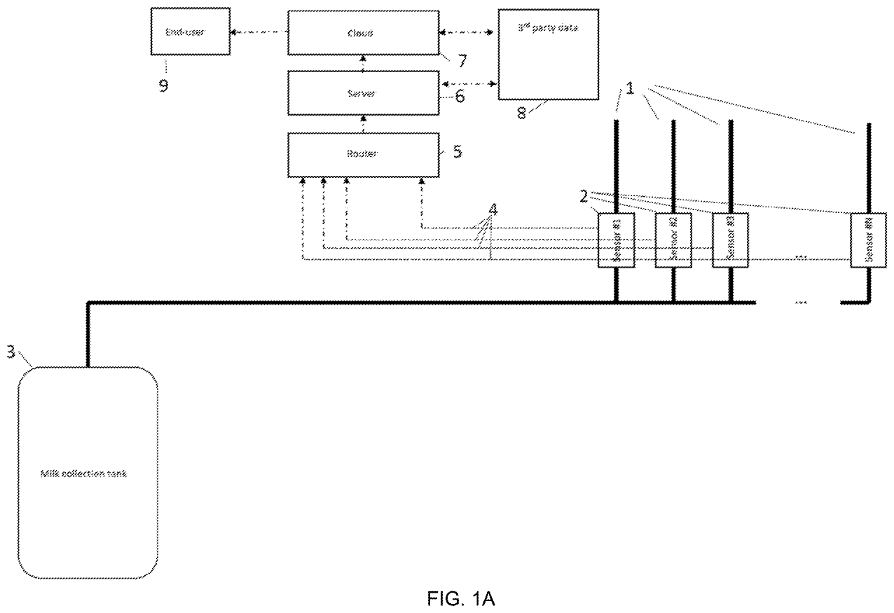

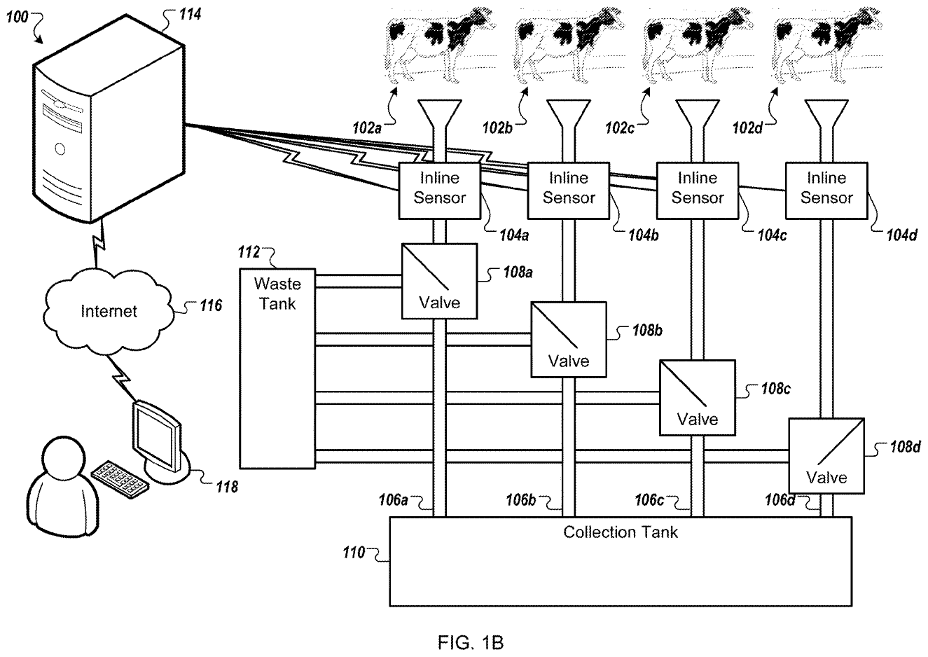

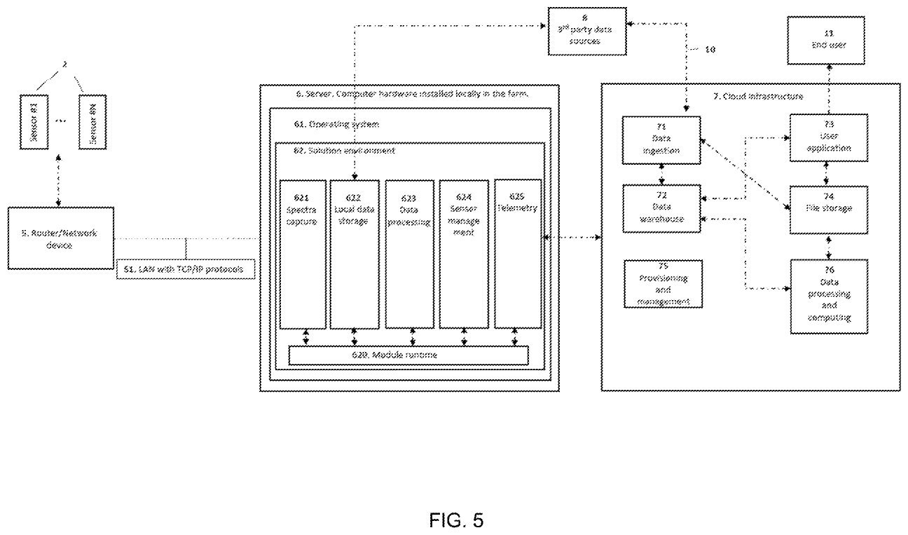

As shown in FIG. 1A, a typical dairy farm can range from few tens of animals to thousands, which results in a large number of animals being milked at the same time several times a day (typically 2-3). Milking stations, designed for milking N cows at the same time are equipped with N, 2N or 4 N sensors (depending on the configuration of the milking system, the milking system can collect milk from all 4 sections of the udder at the same time, or 2 and 2 separately or all 4 separately). As per FIG. 5, the data from each electro-optical sensor 2, comprising milk composition, conductivity, temperature, color, flow, etc., is streamed to a local network device-router 5, which collects the data and sends it to a local data server 6, which is hardware (or software as a virtual machine, etc.), installed locally at each farm. The server 6 comprises operating system 61, for example but not limited to Ubuntu. The operating system contains solution environment 62, which is a virtualized software environment with a module runtime function 620. Module runtime is a platform for running different modules, the modules being custom code applications, designed to perform specific tasks such as spectra capture 621, which connects to sensors, captures data stream, performs data preprocessing such as jitter correction, filtering, averaging, selection of valid data and data conversion to calibrated units such as time to frequency/wavelength, etc., other specific tasks are local data storage 622, where data from the sensor is stored, such as file reports, logs, processed data, partially processed data, as well as data from 3rd party sources 8; data processing 623, where live measurements from the sensor (electric, optical, spectral) are provided to the data model, and aggregated with farm data such as unique animal ID, milking time, historical data, etc. and yield a unique animal milk composition record. Further module is sensor management 624, responsible for connectivity to each of the sensors, monitoring sensor status, performance, and configuration; other module-telemetry 625, responsible for sending and receiving data to and from the cloud infrastructure of the sensor system 7. The cloud infrastructure of the described embodiment serves as a centralized infrastructure, providing resources, components and solutions such as data ingestion 71 component, responsible for receiving data from different farm servers, 3rd party data sources and successfully saving them to cloud data warehouse 72 as a database and/or file storage 74, typically used for large binary files for example spectra, images, external data input etc. Data processing and computing 76 provides cloud-based computing resources for different stages of data processing, where the stage depends on the configuration of the local farm server 6, and the level of local data processed at the farm. Processed data is then again stored in the data warehouse 72 and/or file storage 74, and the data subset is then provided to the user application 73, which in turn provides data to the end user 11 in the form of web application, native mobile application, desktop application etc. The data provided to the end user is adapted for the user in terms of the information content, which may be seen as different selectable views or as interface layers, such as a farmers interface, with more in-depth focus on the herd productivity, farm profitability among others; a veterinary interface with a more focus on individual animal health status, treatment efficacy, historical data, etc.; and a zootechnical interface with a focus on herd selection, allowing the farmer to decide the favorable traits and select the right individual animals for the herd selection to boost the desired traits such for instance high-fat and/or protein content, or disease resistance or milk volume etc. The interfaces are interconnected and allows in-depth monitoring from individual animal to herd to multiple farms, and applying best practices in terms of treatment, selection, nutrition among others based on actual data. The provisioning and management 75 serves as an administrator console for monitoring and configuration of remote farm servers, sensors and entire (Internet-of Things) IoT infrastructure.

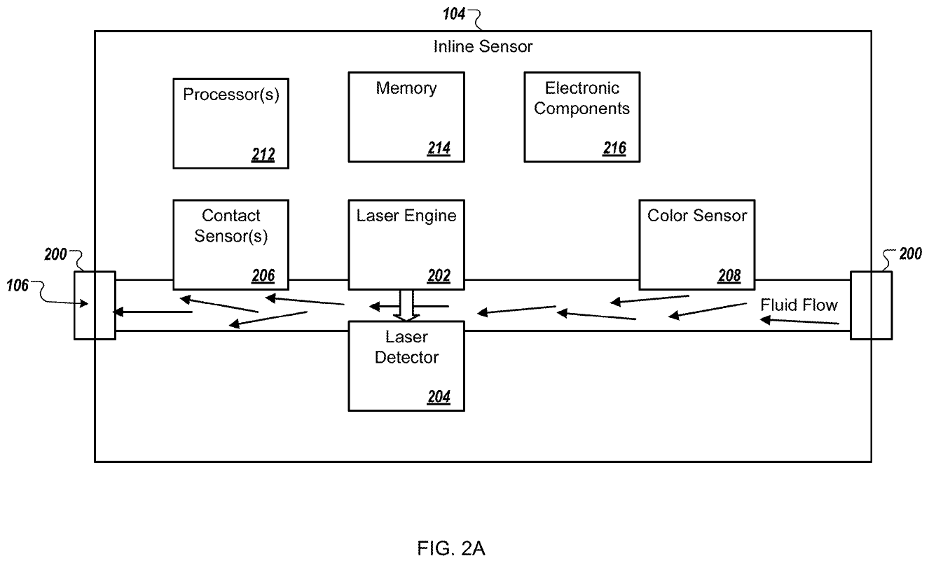

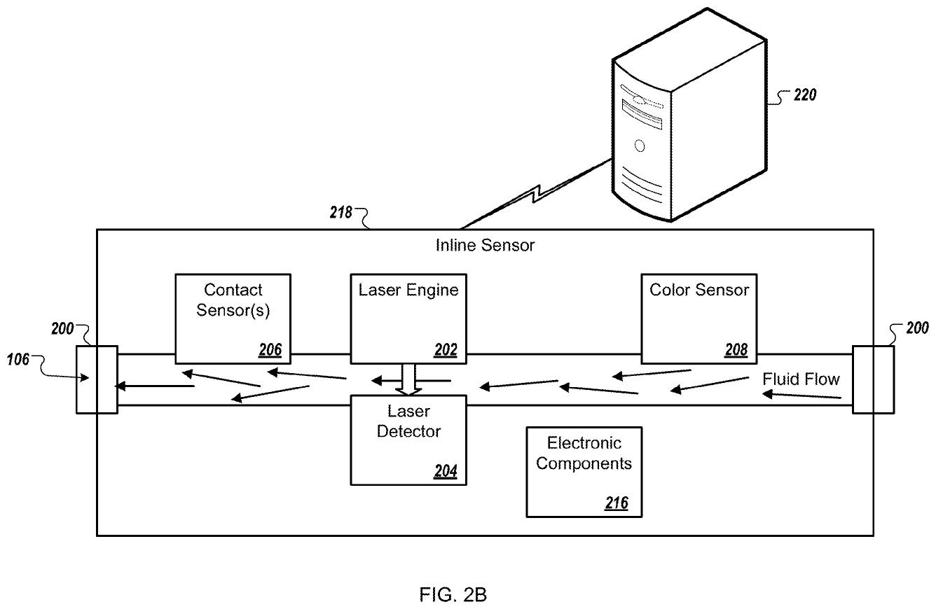

Real-time in-line milk monitoring in dairy farms offers a fairly challenging environment, as the milk flowing through the milking line is not continuous, typically turbulent, contains air bubbles as well as the composition of milk changes dynamically during the milking cycle, the sensor needs to follow the dynamics. For spectral milk composition measurements, the spectrally tunable laser radiation of known intensity and wavelength profile is transmitted through the milk flow in the sensor flow-through compartment via the optical interface and collected by the detector via the respective optical interface. The detector, which is a photodetector—and thus converts the optical signal into electrical, records a time-domain signal (photovoltage or photocurrent), which needs to be processed into a usable absorbance spectrum, that could be used by further data processing to convert the spectral data into the constituent concentration level or in other words-composition. The process of pre-processing the raw data is called spectral capture and is indicated as 621 in FIG. 5. However, in certain configurations, this module may also be run locally at each sensor's CPU module (e.g., processor 212 as shown in FIG. 2A), or in yet another scenario it may also be run in the cloud environment data processing unit 76 as per FIG. 5.

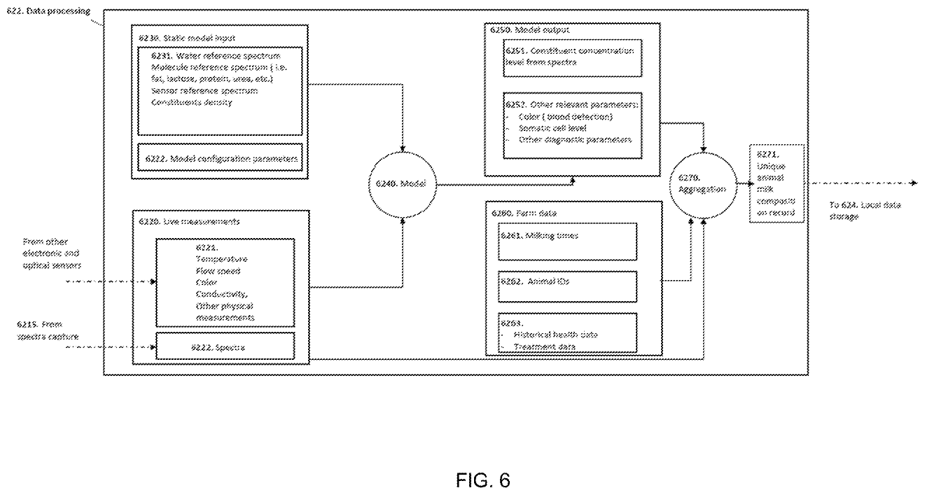

FIG. 6 illustrates spectral capture module 621 in greater detail. Here, the electrical signal from the detector 204 is first amplified by the amplifier 6211, and the current signal is converted to voltage. Amplified signal is then sent to analog-to-digital converter (ADC) 6212 and digitized. The signal from the detector, amplifier and ADC is a time signal—i.e. intensity function as a time. This signal is then sent to the processing device 6214, which maybe a central processing unit (CPU), microcontroller unit (MCU), field programmable gate array (FPGA), complex programmable logic device (CPLD) or a combination thereof, which performs further signal processing such as jitter correction, filtering, selection of valid spectra based on pre-defined rules, averaging and finally, time-to-wavelength/frequency conversion, and this data is then sent to the data processing module 622 via the data link 6215, which essentially is an Ethernet cable. Time to wavelength conversion requires knowledge of absolute wavelength that was emitted by the laser through the milk at all times, this information is then used for the conversion. Frequency tunable laser architecture with absolute wavelength tracking and determination is described in U.S. Pat. Nos. 11,77630 and 11,202,453.

FIG. 1B shows an example system 100 for harvesting milk from livestock using electro-optical inline sensors including tunable laser spectral sensors. In the system 100, cows 102a-d are being milked to harvest the milk. The milk is channeled through inline sensors 104 of pipes 106. Depending on properties of the milk detected with the inline sensors 104, corresponding valves 108 channel the milk to a collection tank 110 or a waste tank 112, for example depending on the presence or absence of contamination such as blood in the milk.

The cows 102a-d represent a milking line through which the milk from one or more farm animals flows during the milking process. A typical dairy farm can range from few tens of animals to thousands, which results in a large number of animals being milked at the same time several times a day (typically 2-3). Milking stations, designed for milking N cows at the same time are equipped with N, 2N or 4 N sensors (depending on the configuration of the milking system, the milking system can collect milk from all 4 sections of the udder at the same time, or 2 and 2 separately or all 4 separately). The milking line may be connected to the animal manually or through a milking robot. The inline sensors 104 can be integrated into the milking line or retroactively added to the milking line between the cows 102a-d and the collection tank 110 and wasted tank 112. The inline sensors 104 can include one or more adapters that facilitate the mechanical interface between the milking line and the inline sensors 104.

Data from the inline sensors 104 is collected over one or more data networks by one or more computing devices 114 such as cloud services or server processes executing on physical or virtual servers. The device 114 can send data or graphical elements over networks such as the internet 116 to be displayed on user devices 118, can store data for long-term storage, or can use the data for other automation processes that engage computer-controlled hardware with computer-readable instructions based on the data. The inline sensors 104 provide electro-optic milk composition sensing in combination with additional physical parameters. As the milk flows through the inline sensors 104, the inline sensors 104 perform analysis (e.g., composition, color, electrical parameters) with optical spectroscopic sensing, color sensing, electrical conductivity, and temperature monitoring and milk volume, milk flow rate measurement. While a single device 114 is depicted, it will be appreciated that the system 100 can use multiple devices to operate as the device 114. For example, one or more local servers can be operated at the harvesting location, one or more remote servers can operate in a data center that is in a different geographic location, etc. In some implementations, a local server receives and pre-processes data received from the inline sensors, and transmits the pre-processed data to an external server (e.g., a cloud-based server). The data is further processed at the external server, and is used to provide feedback to the particular farm based on the data and to aggregate data with additional historical or third-party data to build her data models, as will be further described below. Herd data models are used to understand herd health, and to provide information to make decisions about herd selection, veterinary care, and economic management of the herd. Additionally, the data processing in the external server can determine recommendations for farmers based on the herd data model output (e.g., recommendations about nutrition, treatment efficacy, disease or health warnings, and selection).

The system 100 can allow for automated data collection and control of hardware involved in the milk-harvesting process. For example, the inline sensors 104 can sense milk (or another relevant fluid) as the milk travels through the pipes. As will be appreciated, the milk spends some time (e.g., seconds) in travel between the inline sensor 104 and the valve 108. In less time than it takes for the milk to travel that distance, the inline sensors 104—possibly working with the computing devices 114—can analyze the constituents of the milk. As milk flows through the sensor, the light from the sensor—i.e. tunable laser radiation from the spectral sensor, RGB light from the color sensor continuous shine through flowing milk. The spectral sensor, which is responsible for spectroscopic milk composition analysis records real-time milk absorption spectra that are later reconstructed to real-time concentration data. As the milk composition is not constant over the milking cycle, for most cases, such as farm economy (output) management, the real-time concentration levels are averaged over a single milking event to provide average concentration level values of the milk collected from the cow. At the same time, high resolution, temporal data on concentration levels and other physical parameters such as temperature, conductivity, contamination among others, may indicate valuable information such as inappropriate attachment of the milking system to the utter, sudden blood or other contaminant leak, etc. Both high resolution temporal and averaged data are collected and stored for further processing and aggregation. In an event of contaminant detection, the system 100 can engage a corresponding valve 108 in time to divert contaminated milk to a waste tank 112 instead of allowing the contamination to reach the collection tank 110 and contaminate already-collected milk and/or simply display a warning to the operator and suggest an action that needs to be taken. This is particularly beneficial in cases where contamination is difficult or impossible to detect by human inspection, and where the control of valves is needed at speeds faster than is possible with human reactions. In some examples, the milk travels in pipes 106 for less than two seconds which is sufficient for automated analysis and actuation, but less time than would take for a human to notice blood contaminating the milk, never mind the additional time required to reach and switch a valve to divert the contaminated milk.

The ability to detect contaminants in flowing milk in real-time can allow an automated system to react to contaminated milk quickly and can provide timely warnings in a non-automated system. If undetected, blood leakage into the milking line caused by one animal may contaminate the entire collection tank and thus result in a loss of milk and thus revenues for the farm. Timely detection allows to either automatically shut off or switch the milk from the bleeding animal from the tank to waste, without affecting the milk collected from other farm animals. In farms, where operation is manual, the sensor may provide instant audio or light signal for the operator, so that the milk from the bleeding animal would not reach the milk collection tank.

Similarly, the use of inline sensors capable of sensing properties and contaminants in flowing milk, as opposed to a sensor requiring test samples being drawn and isolated from the regular flow, can be used to identify contamination when it begins. Consider, for example, a system that extracts a test sample every five seconds from a pipe. In this five second window, new contaminants may show up in the milk flow and have four seconds to reach a valve—more than the two seconds required. In this way, the inline sensor 104 can provide for continuous sensing and faster automation than periodic sensing, leading to preserving the milk already collected in the collection tank 110 in a scenario in which periodic sampling would result in contamination in a collection tank, which would contaminate all collected milk and require destruction of the contaminated milk in the collection tank and thus overall lower efficiency due to avoidable wasted material. In addition, periodic sampling is labor intensive and wastes milk as it is an offline measurement as opposed to inline non-invasive optical sensing.

FIGS. 2A and 2B show example devices for sensing phenomena of fluids. In FIG. 2B, a schematic view of the inline sensor 104 is shown. As displayed, the inline sensor 104 includes a fluid channel 106 that can couple to pipe 106 to allow fluid to pass through the inline sensor 104. The channel 106 can include couplers 200 (e.g., threaded or tapped connectors) for connecting in-line with other pipe segments of the pipe 106. Fluid (e.g., milk) flows through the inline sensor 104. As will be appreciated, a certain amount of turbulence in the flow may be present.

The inline sensor 104 includes a number of sensing elements that are used to sense physical phenomena of the fluid such as optical spectra in transmission, in reflectance, temperature,, electrical properties, flow speed, etc.

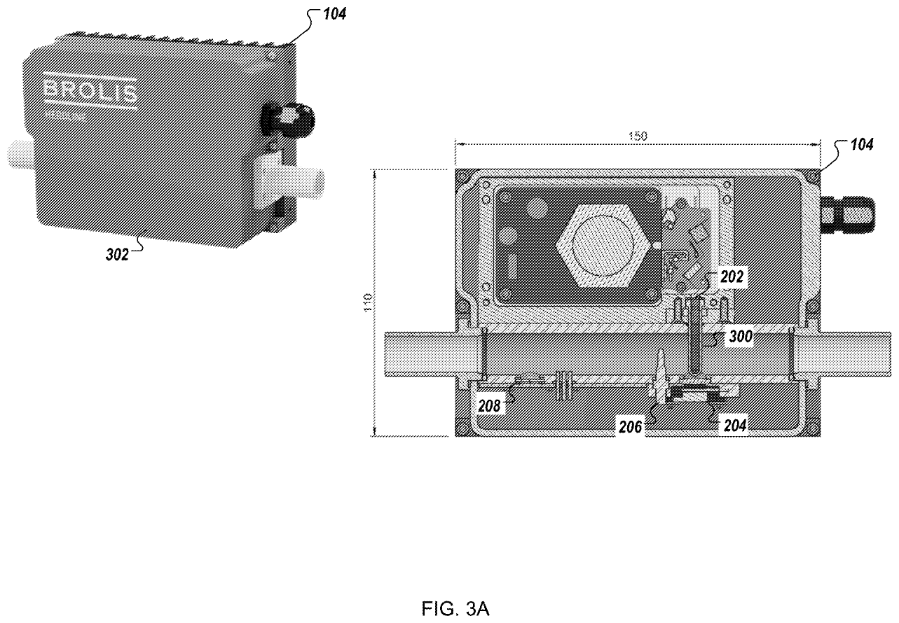

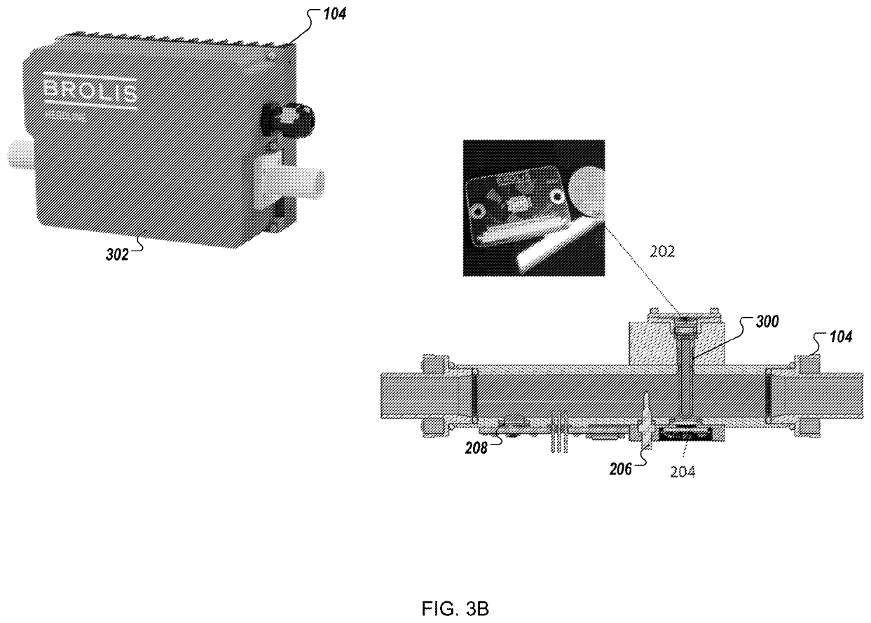

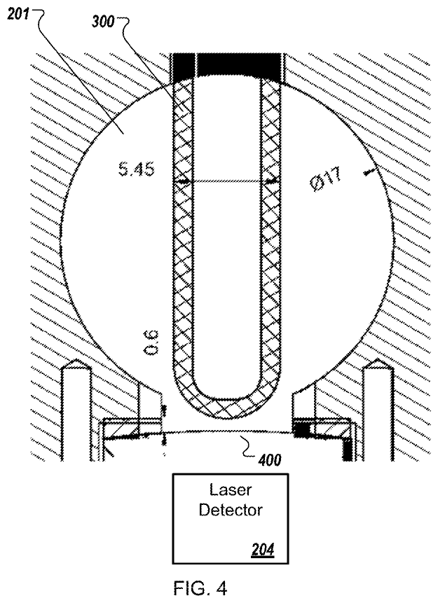

A laser engine 202 emits laser radiation through the fluid as the fluid flows through the sensing system. The laser engine 202 can include a spectrally tunable semiconductor laser or laser array, semiconductor laser based external cavity spectrally tunable laser, and/or a hybrid group III-V/IV semiconductor-based laser or laser spectrometer on a chip. The laser engine 202 is coupled to a probe or sheath (described below in FIGS. 3 and 4) forming an optical interface. The optical interface guides light emitted by the laser to illuminate the milk flow past the sheath or probe. The optical interface maybe an optical fiber, optical lens, system of lenses and optical mirrors, optical window, hollow tube with an optical window, glass tube or a combination thereof. The laser engine 202 provides a laser beam through the optical interface. The laser beam may be collimated, focused or diverging, depending on system optical design. One example of the laser engine could be an external cavity tunable laser based on an III-V semiconductor, such as for example, gallium antimonide, gain-chip, emitting in the 1900-2400 nm band, which covers molecular absorption spectra of lactose, milk fat and milk protein. Another example of the laser engine could be a hybrid gallium antimonide and group-IV semiconductor photonic integrated circuit-based laser spectrometer on-chip, comprised of one or more widely tunable hybrid III-V/IV lasers or laser arrays. For liquid substances other than milk, other III-V semiconductor-based tunable lasers or laser arrays may be considered, depending where the absorption features of the targeted analyte lie within the electromagnetic spectrum. For instance, gallium arsenide may be the material platform of choice for wavelengths 800-1100 nm, indium phosphide for wavelengths 1300-1700 nm, GaSb-beyond 1700 nm.

In some implementations, the laser engine 202 is a solid-state laser-based device having a solid-state gain medium based on a, widely tunable laser for emitting light. In some implementations, the laser engine also includes a wavelength shift tracking device for tracking a wavelength shift of the emitted light. In some implementations, the laser engine also includes an internal absolute wavelength reference which contains a known calibrated spectrum (e.g., an absolute wavelength etalon). In some implementations, the laser engine 202 performs a wavelength sweep, and uses the wavelength shift tracking device in combination with the absolute wavelength reference to provide absolute wavelength determination of the emitted spectrum during the sweep and provide calibration of the emitted spectrum. This wavelength calibration can also include additional components of the laser engine 202, including optical elements such as retro-reflectors, mirrors, prisms among others that enable tunable laser radiation, output beam pointing and spatial stability, as necessary for the application. In some implementations, the laser engine 202 provides an internal calibration of the wavelength for the spectroscopic measurement. Additional details of laser engine components, manufacturing of laser engines, and wavelength calibration hardware that can be used in the sensor system described herein can be found in U.S. patent application Ser. No. 16/609,355, filed May 21, 2018, and U.S. patent application Ser. No. 16/965,867, filed Jan. 31, 2019 and now issued as U.S. Pat. No. 11,177,630, the contents of which are incorporated herein by reference. The laser engine provides tunable laser radiation, which is known at all times with internal wavelength calibration during every spectral sweep across the laser output bandwidth. This information is read by the electronics and used for temporal spectra conversion into frequency—i.e. time to frequency conversion. After preprocessing such as jitter correction, filtering, averaging, and time-to-frequency conversion, such spectra can be used for spectroscopic compositional analysis by the data algorithm.

The light emitted from the laser engine 202 and transmitted through the optical interface illuminates the milk flowing past the probe or sheath and is collected by a laser detector 204 positioned at the channel opposite the optical interface. The laser detector 204 receives the laser radiation after the laser radiation has passed through the fluid and to generate corresponding laser-readings. The laser detector 204 can include a photodiode or photodetector or photodiode or photodetector array with an appropriate optical interface, which may be an optical window, a lens, or a fiber among others. The photodetector or a photodiode is typically a semiconductor-based component, chosen to be spectrally sensitive for the appropriate spectrum of the laser radiation emitted by the laser engine. In some implementations, the detector is AlGaInAsSb/GaSb based PIN or pBp or nBn or superlattice based detector. Especially for wavelength>1700 nm. It may also be an extended GaInAs/InP photodetector. For shorter wavelengths —Si, GalnAs/InP, Ge may also be used.

The distance between the end of the optical interface and the laser detector 204 defines the optical path and is chosen to provide the optimal signal to noise ratio which depends on the optical configuration—i.e. spectral range of the laser, laser output power, measured fluid properties (e.g., absorption coefficient) and detector sensitivity among others. As described below in FIGS. 3 and 4, the optical path is defined in such a way, that when the milk is flowing through the pipe, the laser optical interface 300 and the detector optical interface define a gap of known dimension, which is the thickness of the flowing through liquid, in particular, milk, through which the light passes prior being detected by the detector. The interface is designed so that its mechanical implementation does not obstruct the flow of milk, or the obstruction is minimal. For instance, it could be realized as a glass tube, a hollow tube with a window or a lens among other variants.

The use of the laser engine 202 with an optical interface placed within the flow path of the milk through the channel may require periodic cleaning of the optical interface to ensure transmission of light through the optical interface. Fat residuals from the milk can be deposited on the optical interface (as well as on sensors described below). The accumulation of the fat residuals can in some cases be monitored by the sensors and removed or offset within the collected data. The sensor system is hermetic and resistant to the milking system washing cycles that include high temperature cycling with water, low-pH and high-pH cleaning agents. Additionally, the system may include alerts when the residuals deposited on the optical interface reach a threshold level that threatens the accuracy of the measurements. In some implementations, one or more sensor systems including redundant laser engines, laser detectors, and other sensors can be included inline to ensure accurate readings and measurements.

A contact sensor group 206 contacts the fluid as the fluid flows through the inline sensor 104 and senses one or more contact-phenomenon of the fluid to create corresponding contact-readings. The contact sensor group 206 can include a temperature sensor for measuring temperature of the milk and/or a pair or an array of electrodes for measuring electrical conductivity of the milk. The electrical conductivity value and dynamics are associated with a concentration of somatic cells present in the milk flowing through the sensor. Additionally, conductivity, temperature or both combined may also be used to evaluate flow of the milk passing through the sensor, which may be helpful for the quantitate evaluation of the milk constituent concentration level with the laser engine 202. In some implementations, the contact sensor group 206 includes multiple discrete sensors positioned apart from one another in or adjacent to the fluid channel. The contact sensor group 206 can include an electrical conductivity sensor and a temperature sensor. In other implementations, the contact sensor group 206 includes a single sensor capable of detecting multiple contact-phenomena, or alternatively, includes multiple sensors positioned adjacent to one another.

An optical emitter within the color sensor 208 emits a non-coherent light into the fluid, reflected light from the fluid is collected by and RGB detector, forming a color sensor. A color sensor 208 can receive the non-coherent light after the non-coherent light has passed through the fluid and to generate corresponding non-coherent-readings. The optical emitter of 208 can emit in the wide-spectrum light into the milk flowing past the emitter. Light is reflected from the milk flow and collected by the color sensor 208. The color sensor 208 can include an optical window, wavelength filter, optical lens, optical prism, diffraction grating or a combination thereof. Blood presence in milk will affect the color of milk, which in turn will change the reflectance spectrum of the light and be detected by the color sensor 208. In some implementations, milk fat content may also be discriminated by the change of the reflection spectrum-essentially, color-as collected by the color sensor 208, and may be used in combination with the spectral engine data or stand-alone for milk fat level evaluation.

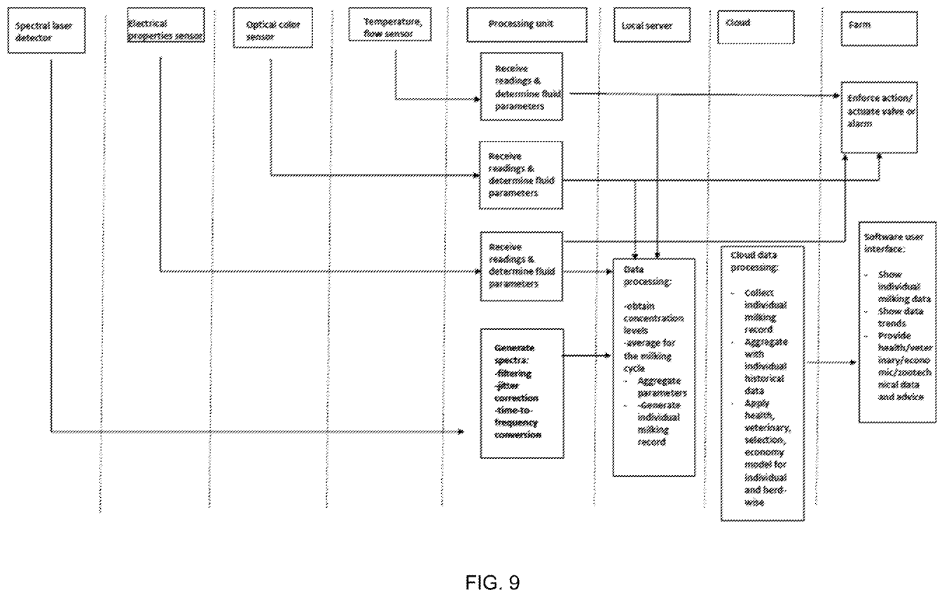

The inline sensor 104 can include computational hardware such as one or more processors 212, memory 214, and other electrical components 216. Examples of processors, memory, and other electrical components are described in greater detail below, for example, with respect to FIG. 9.

FIG. 2B shows another example inline sensor 218. In the inline sensor, some or all of the computations performed by processors and memory are performed by 220 a computing device 220, which may include one or more servers, desktop computers, etc.

FIGS. 3A and 3B show example inline sensors 104 for sensing phenomena of fluids. Shown here are isometric views and a cut-away view of the inline sensor 104. A housing 302 is shown that contains the components (illustrated in FIG. 2B), including the laser engine 202, the laser detector 204, the contact sensor group 206, the optical emitter and color sensor 208, and electronic components such as the processors 212, the memory 214, and other electronic components 216.

In the cutaway view, the contact sensors group 206, laser engine 202, laser detector 204, and optical emitter and color sensor 208 are shown. As shown, the laser engine 202 includes a sheath 300 that encases an optical interface of the laser 212 to prevent the fluid from reaching and coming into contact with the laser engine 202. or example, the sheath 300 can be constructed from glass, plastic, stainless steel, or another appropriate material that allows the laser radiation of the laser engine 202 to pass through the optical interface within the sheath 300 to the laser detector 204. Depending on the material (e.g., for non-transparent material), the sheath 300 can include one or more optical windows of transparent material. Sealing structures (e.g., adhesive layers, slip fits, gaskets) can be used to provide a fluid-proof/water-proof seal from external humidity. The contact sensor group 206, laser engine 202, laser detector 204, and optical emitter and color sensor 208 210 sense multiple parameters of the flowing fluid, including a temperature of the fluid, conductivity of the fluid for composition determinations, and color of the fluid based on reflected mode detection. The laser engine as shown in FIG. 3(A) is external cavity laser with III-V semiconductor gain-chip embedded in Metcalf-Littman external cavity configuration with a rotating mirror for wavelength tuning. Other variants of the cavity configuration could include Littrow, or may use micro-mechanical membrane mirror (MEMS) instead of the rotating mirror. FIG. 3(B) illustrates another possibility, where the laser engine 202 is realized as a hybrid III-V/IV laser spectrometer-on-chip, where wavelength discrimination is achieved electronically, without moving parts, by means for example, using a Vernier-type filtering technique, among others.

FIG. 4 shows an example fluid channel 106 in a side view. As shown here, the sheath 300 is shown positioned inside the fluid channel 106. The fluid channel 106 is circular in cross-section, although other geometries may be possible. The circular cross-section of the fluid channel 106 encourages laminar flow of the fluid through the channel. In some implementations, the fluid channel 106 is 20 mm, 30 mm, 40 mm, 50 mm, 60 mm, or 80 mm in diameter, or any other suitable diameter. As described above, the optical interface sheath 300 is formed as a needle which does not obstruct the flow of the fluid through the fluid channel 106. In some implementations, the optical interface sheath includes a window of material which is allows transmission of the spectrally tunable laser radiation of a known intensity and wavelength profile through the window and through the milk flow in the channel 106 to the laser detector. In some implementations, the optical interface sheath is formed as a needle into the fluid channel, using a material which is opaque for the body of the sheath and a laser radiation transparent window. In some implementations, the optical interface sheath has other configurations, including as a tube/needle extending further or less far into the channel, as a bar across the channel, or as a window in a side wall of the channel, or any other suitable configuration.

The laser engine 202 and laser detector 204 are fixedly held at least partly in the fluid channel 106 such that a portion of the fluid flows between the laser engine 202 and the laser detector 204. In particular, a portion of the fluid flows between the optical interface in the sheath 300 and the laser detector 204. In this example, the laser engine 202 (i.e., the optical interface within the sheath 300) and a biconvex lens 400 are held at a distance of 0.6 mm from each other, allowing a flow of fluid between for sensing. The lens 400 can be used, for example remove coherence from the laser emission before it reaches the laser detector 204. The end of the sheath 300 can have a width determined by the type of fluid to be sensed, with larger widths used for some fluids which are more transparent. As will be appreciated, the distance can depend on fluid type (and its absorption properties) and laser output power. Longer distances can allow for more interaction with target analyte molecules and thus smaller concentrations/higher accuracy can be obtained. In some implementations, an end of the sheath 300 for sensing of milk can be 0.25 mm, 0.5 mm, 1 mm, 1.5 mm, 2 mm, 2.5 mm, 3 mm, 3.75 mm, 8 mm, 10 mm, 12 mm, 15 mm, or any other suitable distance from the laser detector 204. Other distances are possible, including greater or shorter distances. Because milk contains predominantly water, the measurement of fats, protein, and lactose requires knowledge of a precise transmission distance between the optical interface of the laser 300 and the optical interface of the laser detector 204, which, essentially is the thickness of the milk flowing through the laser light path. If the gap between the components is too large, the signal from the water component of the milk will dominate the sensed spectra and the valuable modulation of the laser spectra by the targeted constituents (lactose, fats and protein) will be drowned in the noise. On the other hand, if the gap between the components is too small, the absorption constituents such as lactose and protein will also be small and difficult to measure, as the laser intensity modulation by the milk constituents is directly proportional to the molar absorptivity of the target molecules, which in turn modulate the laser spectrum. Too small gap will lead to a small milk volume between the laser and the detector, and thus too small signal modulation to be detected by the detector. Optimal gap depends on the laser output power level, flowing through fluid properties and the detector sensitivity and is thus chosen for optimal signal to noise ratio, based on the component performance, comprising the system. This step is done at the assembly and factory calibration.

The distance between the end of the sheath 300 and the laser detector 204 is oriented toward a bottom of the fluid channel 106, such that the portion of the fluid channel 106 between the end of the sheath 300 and the laser detector is always full of fluid due to gravity. Any turbulence of the fluid resulting in air bubbles or other artifacts can be detected from the photo-detection data at the laser detector 204 and removed from the data using appropriate algorithm during signal processing.

Real-time in-line milk monitoring in dairy farms offers a fairly challenging environment, as the milk flowing through the milking line is not continuous, typically turbulent, contains air bubbles as well as the composition of milk changes dynamically during the milking cycle, the sensor needs to follow the dynamics. For spectral milk composition measurements, the laser engine 202 emits spectrally tunable laser radiation of a known intensity and wavelength profile, which is transmitted through the milk flow in the channel 106 via the optical interface in sheath 300 and collected by the laser detector 204 via a respective optical interface. The laser detector 204, which can be a photodetector, converts the received optical signal into an electrical signal and records a time-domain signal (e.g., photovoltage or photocurrent). The signal can be processed into a usable absorbance spectrum and can undergo further data processing to convert the spectral data into the constituent concentration level or milk composition.

In an example, the electrical signal from the laser detector 204 is first amplified by an amplifier, and the current signal is converted to voltage. Amplified signal is then sent to an analog-to-digital converter (ADC) and digitized. The signal from the detector, amplifier and ADC is a time domain signal-i.e. intensity function of time. This signal is then sent to the processing device (e.g., a central processing unit (CPU), microcontroller unit (MCU), field programmable gate array (FPGA), complex programmable logic device (CPLD), or any combination of these devices or another device or devices, which performs further signal processing such as jitter correction, filtering, selection of valid spectra based on pre-defined rules, averaging and finally, time-to-wavelength/frequency conversion, and this data is then sent to a data processing module via a data link (e.g., an Ethernet cable). Time to wavelength conversion requires knowledge of absolute wavelength emitted by the laser radiation through the milk at all times, this information comes from the laser engine 202 and is then used for the conversion.

FIG. 5 shows IT hardware and software architecture that can be used. For example, the hardware architecture shown in FIG. 5 can be used in a milk harvesting operation using inline sensors. Elements of FIG. 5 are described previously

FIG. 6 shows processing 622 to generate unique animal milk composition records. Input is received over 6215 including data from spectra capture and other electronic optical sensors is received and used as live measurements 6220. This input 6215 can include data 6221 including temperature, flow speed, color, conductivity, as well as other physical measurements. This input 6215 can include spectra data 6222.

The processing 622 also uses static model input 6230 along with the live measurements 6220 in a model 6240. The static model input 6230 can include model reference parameters 6222 and reference data 6231 which can include water reference spectrum, molecule reference spectrum (such as for fat, lactose, protein, urea, etc.), sensor reference spectrum, and constituent density data.

The model combines the data 6220 and 6230 to create model output 6250. The model output 6250 can include constituent concentration levels from spectra 6251 and other relevant parameters 6252 such as color (e.g., for blood detection), somatic cell levels, and/or other diagnostic parameters.

Aggregation 6270 can use the model output 6250, the live parameters 6220, and farm data 6260 to create unique animal milk composition records 6271 to be sent to local storage 624. The farm data 6260 can include data related to farm activities (or other operational activities for other types of operations), including milking times 6261, animal identifiers 6262, and data 6263 such as historical health data and/or treatment data.

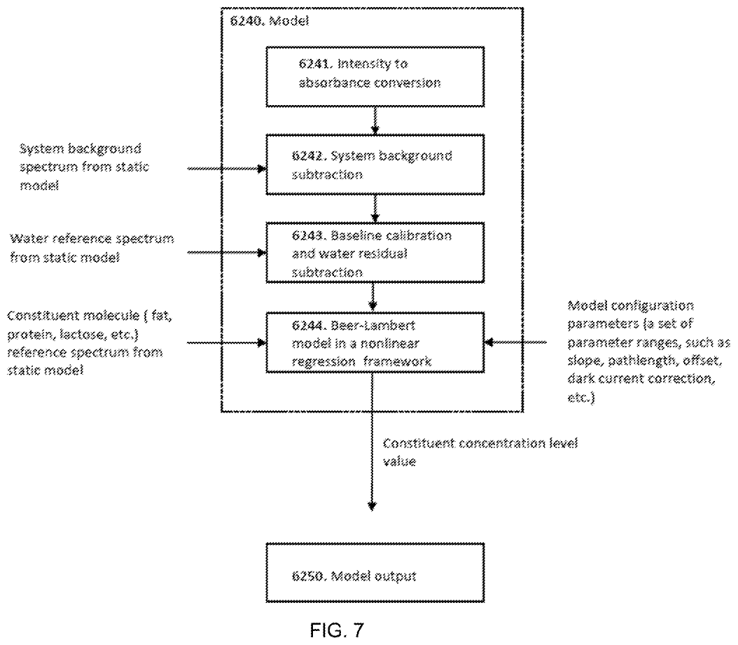

FIG. 7 shows an example of the model 6240 in greater detail for composition analysis. In this example, the model 6240 operates to generate the model output 6250.

Laser intensity values, as detected by the photodetector are converted to absorbance in step 6241. Then, system background is subtracted in step 6242. One example could be such that the system background spectrum is provided from the static model. System backgrounds all optical features of the system—i.e. laser, photodetector, optical elements, mirrors etc., except for the spectrum milk or other liquid flowing through the sensor.

System background subtraction is performed 6242, and the resulting spectrum is then mainly the optical spectrum of the fluid flowing through the spectrum. For example, milk, which is a substance of a number of different molecules—water, proteins, fats, lactose, metabolites and different analytes. Following step is baseline calibration and water residual subtraction in step 6243. Water is the main baseline dominant and thus is important to subtract in order to uncover other constituents of the fluid.

After the baseline calibration and water residual subtraction is performed 6243, target constituent concentrations are derived using, for example, a Beer-Lambert absorption model in a nonlinear regression framework in step 6244. For higher accuracy, reference spectra for constituents are provided from the library within the static model. Absorption spectra change in terms of peak width, slopes, etc as concentration and temperature changes. And static model input minimizes the prediction error. Additional parameters or parameter ranges of the nonlinear model may be slope, pathlength, offset, detector background correction, dark current correction among others. The output of the step 6244 is already a quantite concentration level of the target constituent or constituent set. The value is sent to the model output 6250 and included in the single milking data set with additional parameters from other sensors and 3rd party data.

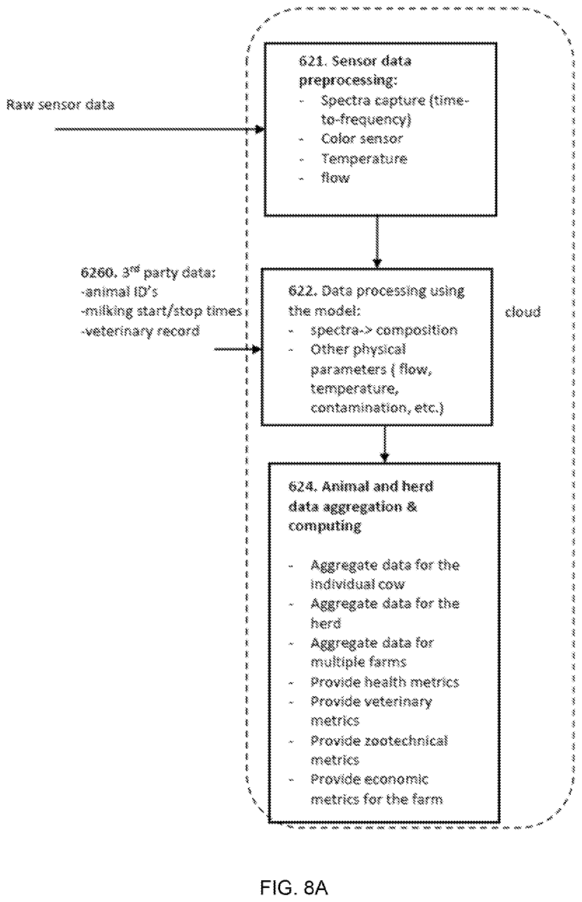

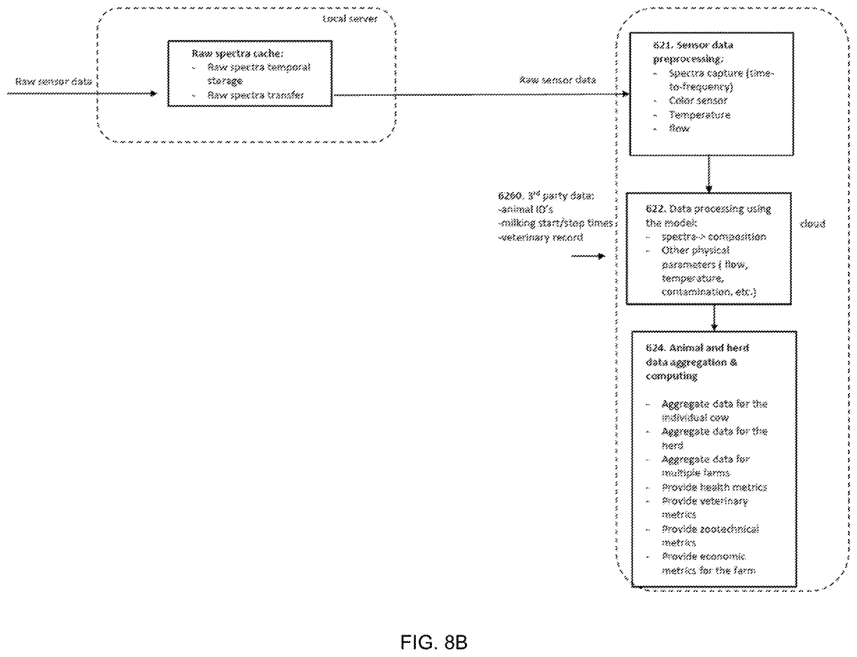

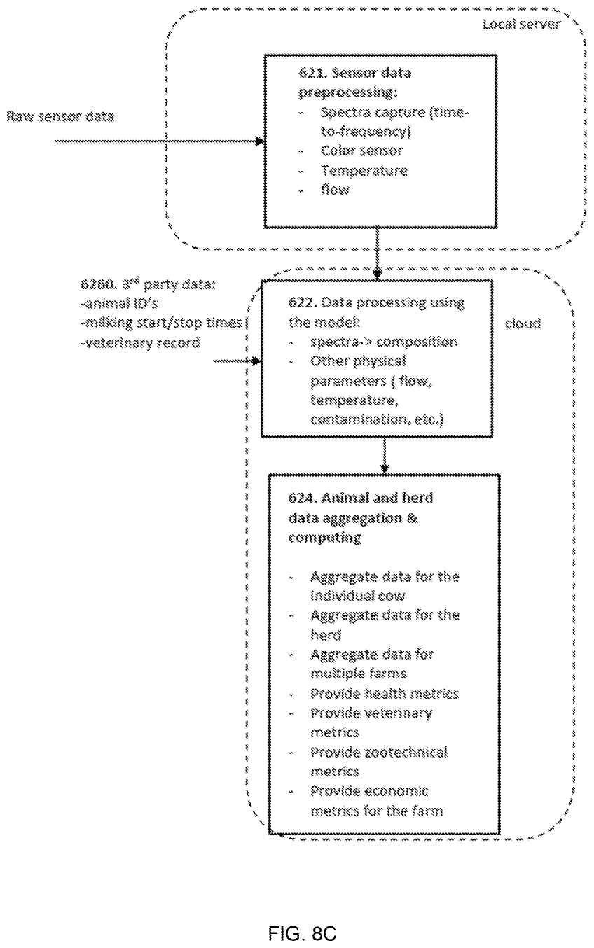

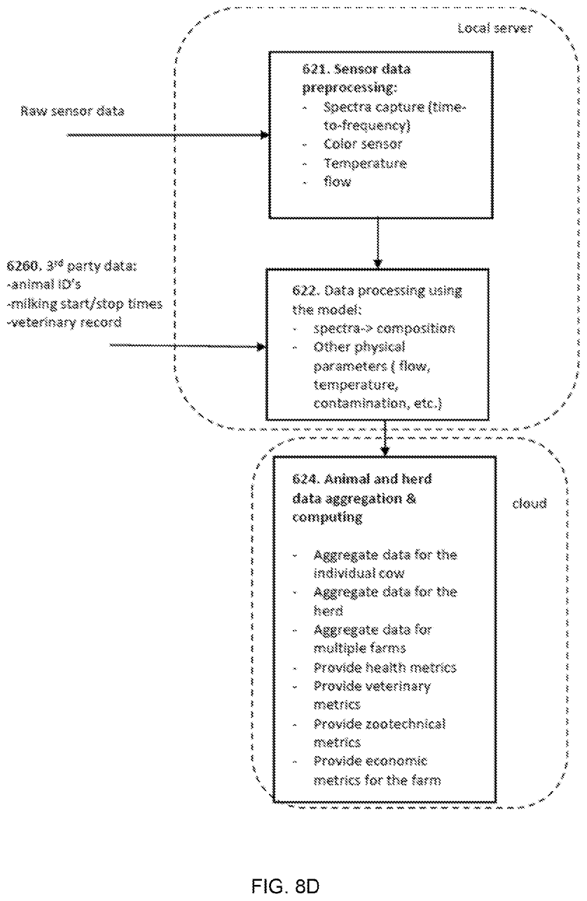

FIG. 8A-8D show generation of data from raw sensor data. Sensor data 621 is processed, using the raw sensor data as previously described. This processing 621 can generate spectra capture (e.g., time-to-frequency formats), color sensor data, temperature, flow, and other parameters. Third party data 6260 such as animal identifiers, milking start/stop times, and veterinary records can be incorporated in data processing using the model 622, which can include spectra to composition determinations, and other physical parameter determinations such as flow, temperature, contamination, etc.

Animal and herd data aggregation and computing is performed 624. This can include aggregating data for an individual animal (e.g., cow) and/or for the herd of animals. Data can be aggregated across multiple farms. This can be used to provide health metrics for individual milkings, individual cows, farms, or farm groups. Veterinary metrics, zootechnical metrics, and economic metrics can also be created.

In one example shown in FIG. 8A, operations 621-624 are performed in cloud computing architecture. In one example shown in FIG. 8B, a raw spectra cache is used to cache raw sensor data with raw spectra temporal storage and raw spectra transfers. In one example shown in FIG. 8C, operation 621 is performed on a local server (e.g., physically located on a farm) and operations 622 and 624 is performed in cloud architecture. In one example shown in FIG. 8D, operations 621 and 622 are performed on a local server while the operation 624 is performed in the cloud.

FIG. 9 is a swimlane diagram of an example process for sensing fluid. In the process, a processing unit receives readings and determines fluid parameters from sensors such as an electrical properties sensor, optical color sensor, and temperature flow sensor. The processing unit also generates spectra including filtering, jitter correction, and time-frequency conversions. A local server performs data processing including obtaining concentration levels and averaging the levels for a milking cycle. This can produce aggregate parameters (e.g., for all cows on a farm) and individual milking records for a single milking session of a single cow.

Cloud data processing can include collecting individual milking records and aggregating the individual milking records with historical data. Health, veterinary, selection, and economic models can be provided for individual and herd-wise analysis.

Farm equipment can enforce actions/actuate valves or alarms based on the optical color sensor and/or temperature sensor data and/or electrical properties sensor. A software user interface can be used to show individual milking data, show data trends, and/or provide health/veterinary/economic/zootechnical data and advice.

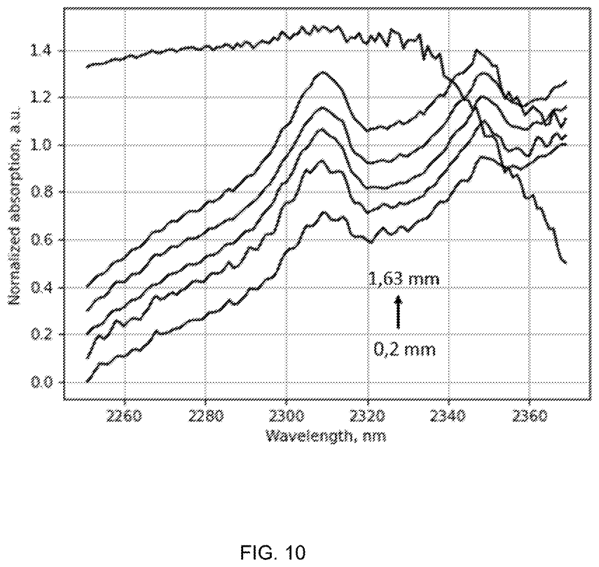

FIG. 10 shows data illustrating absorption spectra of milk for a variety of transmission path distances measured between the optical interface and laser detector. FIG. 10 shows six spectral absorption signals over a variety of wavelengths of light for transmission paths between the optical interface and photodetector. From the bottom line to the top line, the absorption spectra are shown for transmission paths of 0.2 mm, 0.4 mm, 0.66 mm, 0.82 mm, 1.11 mm, and 1.63 mm. FIG. 10 illustrates the spectral data for various pathlengths (i.e., gap between optical sheath interface and laser detector) at a fixed laser power. The peak-to-valley ratio of the milk spectra is related to the measurement specificity, and the pathlength (gapsize) should be chosen such that the peak-to-valley ratio is the highest. If the gap between the optical interface (or the end of sheath 300 in FIGS. 3 and 4) and the laser detector 204 is too small, the absolute absorption of the milk fat with be also small, resulting in a weakly modulated laser signal as maybe seen for gap of 0.2 and 0.4 mm for the given laser power and spectral region. The milk spectra data shows an optimal for gap of 0.6-1 mm, where the laser signal modulation by molecular absorption is highest. This can be seen from the peak-to-valley ratio of the two fat peaks. For larger gap widths, at a given laser output power, water absorption becomes dominant, and the signal is lost. For different applications, and different implantation configurations, the gap will vary from tens of microns, through millimeters and, in some cases, centimeters or meters, depending on the laser output power used in the sensor and the optical properties of the fluid in the line.

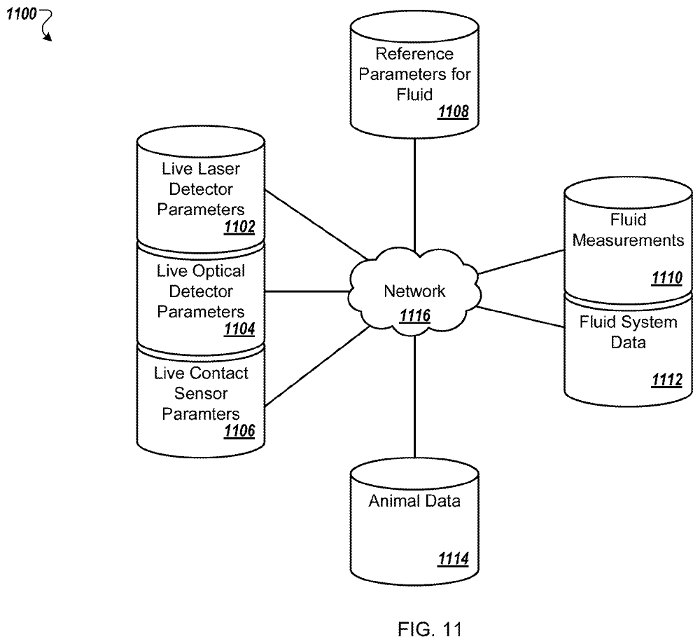

FIG. 11 shows example data 1100 usable in sensing fluids and controlling automated devices. In this example, the data is held in various datastores 1102-614 and is accessible over a network 1116. The datastores 1102-614 can in some cases be embodied in one or more computing elements (e.g., servers, virtual machines, desktop computers, hand-held devices) and in one or more data structures (e.g., relational databases, files, data messages, memory entries) in the one or more computing elements. The network 1116 can include internal data buses, external data networks, etc.

The live laser detector parameters 1102 can include incoming data and historic stored data generated with the laser detector 204. For example, the laser detector 204 can generate analog data that is converted to digital data, conditioned (e.g., removing or reducing jitter, filtering, averaging, converting to aggregates or smaller data formats), and transmitted over the network 1116 for storage. The laser parameters 1102 can include time-series data comprising spectra values and corresponding timestamp data, or another appropriate format.

The live optical detector parameters 1104 can include incoming data and historic stored data generated with the color sensor 208. For example, the color sensor 208 can generate analog data that is converted to digital data, conditioned (e.g., removing or reducing jitter, filtering, averaging, converting to aggregates or smaller data formats), and transmitted over the network 1116 for storage. The optical parameters 1104 can include time-series data comprises light intensity values, color values, or other values and corresponding timestamp data, or another appropriate format.

The live contact sensor parameters 1106 can include incoming data and historic stored data generated with the contact sensor group 206. For example, the contact sensor group 206 can generate analog data that is converted to digital data, conditioned (e.g., removing or reducing jitter, filtering, averaging, converting to aggregates or smaller data formats), and transmitted over the network 1116 for storage. The contact parameters 1106 can include time-series data comprises temperature, conductivity values, flow speed, or other values and corresponding timestamp data, or another appropriate format.

Reference parameters for fluid 1108 can include a library of reference values for a particular fluid (e.g., milk) or variety of fluids. For example, for a fluid with various components (e.g., milk) reference parameters 1108 can be stored for each component or likely contaminant (e.g., water, milk protein, lactose, urea, blood). These reference parameters can be used, for example, when generating quantitate output such as milk constituent concentration level acquired from the spectral measurements and other relevant parameters, such as temperature, milk volume, milk conductivity, somatic cell count, blood content, etc., which may be stored as fluid measurements 1110.

Fluid measurements 1110 can include both individual and aggregate measurements. For example, individual measurements may include measures of the fluid at a specific point in time or within a small time windows (e.g., less than half a second). The individual measures can provide a “snap-shot” of the fluid at a given moment. On the other hand, aggregate measures can be created to capture properties of fluid over a longer period of time. For example, a milking session for a given animal may have a single set of aggregate measurements 1110. The aggregate measurements may be created by aggregation of individual measurements. For example, hundreds or thousands of fluid measurements recording fat content in milk can be aggregated into a mean fat content for the milking session. As will be appreciated, this aggregation may include all individual measurements within a time, or may include a subsampling of all individual measurements. Use of both individual and aggregate measurements can provide a number of advantages. For example, fat content of milk can be expected to change over the span of a milking session. The fat content at any particular point may be of limited value, and thus taking a mean of individual fat content measurements can provide for a more useful measure to the operation of a dairy farm, when aggregating the milking values across longer periods of time—i.e., weeks, months. Such composition trends reveal valuable insights into individual animal health, lactation, nutrition, recovery (treatment efficacy) and allow early-stage action for disease onset, nutrition change etc. In some example, hundreds or thousands of individual measurements are created per second, and aggregating them into fewer aggregate measurements can allow for reasonable understandability. On the other hand, many automated systems can operate at hundreds or thousands of cycles per second, and having the individual measurements in addition to the aggregate measures can allow for accurate and responsive control systems. In some cases, keeping hundreds or thousands of individual measurements per second can allow for pinpointing a time in which a change in a fluid is first noticed, which can aid in troubleshooting a faulty system (e.g., a milking cup coming lose on a cow). Similarly, time-series data of fluid measurements can be used to identify environmental conditions that impact fluid production. For example, changes to light, sound, temperature, or other environmental factors can be recorded with light, sound, temperature, or other environmental sensors around cows being milked. These environmental factors can be compared to individual and/or aggregate to determine improved environmental conditions for milk production (e.g., modifying light to follow daylight intensity, reducing abrupt sounds, increasing heat with a heater).

Fluid system data 1112 can include information collected or generated for the operation of a fluid system (e.g., system 100). The data 1112 can include computer-readable instructions for various components of the fluid system that can be edited or used to drive automated devices (e.g., valve 108). In addition, records of historic operation (e.g., information about milk harvesting including volume, timelines of harvesting, operation of the vales 108, and volume currently in collection tank 110) can be stored in the data 1112.

Animal data 1114 can record information about animals used in the fluid system (e.g., the cows 102). This animal data 1114 can record individual animal information (e.g., indexed by a unique identifier associated with each animal) and aggregate information about a herd of animals, such as historic health data, lactation data, treatment data etc. The animal data 1114 can also be used to provide timely alerts to herd management professionals regarding possible health issues present in individual animals before the health issue spreads within the herd. Timely health management ensures minimal downtime of the animal as well as minimal use of “hard” medication such as antibiotics if the onset of the disease is diagnosed early enough to be treated with lower-cost and lower downtime alternatives, maximizing the output of both individual animal as well as the herd. In addition, animal data 1114 will provide insight into treatment efficacy and may be used as a reference for establishing the “standard-operation-procedure” when treating particular health issue by choosing the medication and/or procedure that provided highest efficacy based on historical herd and/or farm data.

The animal data 1114 recording information related to the milk production, content, and characteristics of individual animals can be used in other aspects of herd management and selection. Knowing milk composition data for every herd animal is an immensely valuable asset for controlled herd selection, where only herd members with desired traits that are aggregated in the milking data would be used for herd selection. For instance, an animal providing lower annual milk volume but milk rich in fats and proteins can be more valuable as compared to another member of the herd which provides higher volume of milk annually as in some countries, a surplus payment for excess fat and protein exists.

Animal data 1114 can be reported and displayed in a variety of interfaces and on a variety of client devices as described herein. Information may be formatted or filtered for particular uses in each interface in a program for storing and visualizing the information, for example a farmer's interface, with more in-depth focus on the herd productivity, farm profitability among others, veterinary interface with a more focus on individual animal health status, treatment efficacy, historical data, etc., and zootechnical interface with a focus on herd selection, allowing the farmer to decide the favorable traits and select the right individual animals for the herd selection to boost the desired traits such for instance high-fat and/or protein content, or disease resistance or milk volume etc. The interfaces can be interconnected and allows in-depth monitoring from individual animal to herd to multiple farms, and applying best practices in terms of treatment, selection, nutrition among others based on actual data. The provisioning and management serve as an administrator console for monitoring and configuration of remote farm servers, sensors and entire Internet-of Things (“IoT”) infrastructure on the farm.

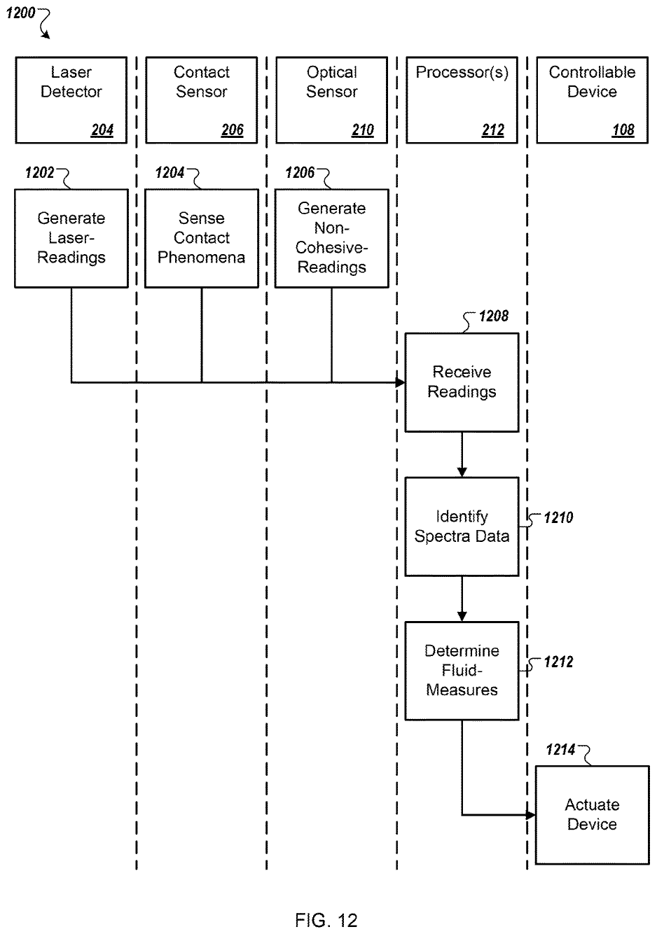

FIG. 12 shows a swimlane diagram of an example process 1200 for sensing fluid usable in a milk-harvesting system. The process 1200 can be performed by, for example, the system 100 and will therefore be described with reference to elements of the system 100. However, another system or systems may be used to perform the process 1200 or another similar process.

The laser detector 204 generates laser-readings 1202. For example, the laser detector 204 receives the laser radiation after the laser radiation has passed through the fluid and to generate corresponding laser-readings. As such, various properties of the fluid will influence the laser radiation as it passes through the fluid, which will thus be recorded by the laser-readings in computer-readable data. In particular, laser signal will be modulated by specific molecular absorption of the milk constituents when passing through the flow. The modulation is later recovered by signal processing and data model and converted to quantitate constituent concentration levels.

The contact sensor group 206 senses contact phenomena, which can include electrical phenomena and temperature phenomena 704. For example, the contact sensor group 206 can sense one or more contact-phenomenon of the fluid to create corresponding contact-readings such that the determining 1212 of one or more fluid-measures can further comprises using the contact-readings. As such, various properties of the fluid will influence the sensing elements of the contact sensor (e.g., a semiconductor thermistor will change resistance strongly depending on temperature), which will thus be recorded by the contact-readings in computer-readable data.

The color sensor 208 generates non-coherent-readings 1206. For example, the color sensor 208 can generate corresponding non-coherent-readings such that determining one or more fluid-measures further comprises using the non-coherent-readings. As such, various properties of the fluid will influence the sensing elements of the color sensor (e.g., based on color, intensity, or other optical properties), which will thus be recorded in the non-coherent-readings in computer-readable data, an example of the color sensor is, for example a standard commercial RGB color sensor such as but not limited to S9706 from Hamamatsu, or AS73211 from AMS AG among others

The processors 212 receive the readings 1208. For example, the processors 212 can access the laser-readings, the contact-readings, and/or the non-coherent-readings. The processors 212 identify spectra data 1210. For example, the spectra data can be identified to be reflective of physical properties of the fluid as the fluid flows through the sensing system.

The processors 212 determine fluid-measures 1212. For example, using the spectra-data and reference-data defining one or more reference spectra for each possible constituent of the fluid, the processors 212 can determine various values for fluid-measures. For example, the processors 212 can access a library of spectra, such as for instance temperature dependent water and constituent reference spectra, which can be stored as part of the firmware of the sensor. The library can also contain system background spectrum, fitting parameter set, parameter fitting range, etc. In some implementations, the parameter set contained in the library includes one or more of the pathlength, offset, slope, and dark current correction associated with the inline sensor set up in the milking system for use in interpreting the collected data. The static model can be updated and corrected for system background spectrum and water reference spectrum during operation on-site, when the milking is being purged with water for washing. This allows timely correction for any of the drifts from the original library data, and provides higher measurement accuracy. This data is used by the model for processing the live measurement data. Here, the collected spectral data undergo preprocessing, and the data in the form of intensity as a function of wavelength or frequency is provided for further processing until the output can provide estimated concentration level value. In an example processing operation, the collected spectral data intensity is first converted to an absorbance, followed by the system background subtraction. Here, the system background spectrum is fed from the static model. This allows decoupling the system related nonlinearities and spectral artefacts from the milk or, in broader terms, object under investigation. Once the system background is subtracted, the next operation is the baseline calibration and main baseline dominant residual subtraction. In case of milk, the main baseline dominant comes from water. The water reference spectrum is fed from the static model, fitted for the best fit and residuals subtracted. Finally, the spectra are processed using, in some embodiments, Beer-Lambert model in nonlinear regression framework. In some implementations, other regression models are used in the data processing. Here, the static model provides reference spectra for the constituents under investigation such as milk fat, lactose, protein, water, etc. in combination with a set of model configuration parameters, such as ranges for offset, slope, path length, etc. that are used together with the reference spectral data to provide the best fit and recover the constituent estimated concentration levels, which is then sent to the model output.

The controllable device 108 actuates 1214. For example, the controllable device 108 can include an automated valve configured to selectively direct a flow of the fluid to a plurality of output channels. The automated valve can be actuating with computer-readable instructions based on at least one of the fluid-measures.

In some cases, engaging the automated valve to direct the fluid to a waste tank is responsive to determining a fluid-measure for a contaminant is greater than a threshold value. For example, the threshold may be set so that very low values of contamination, which are more likely to be the result of noise in the sensing system than actual contamination, will not result in actuation. In some cases, actuating the automated valve based on at least one of the fluid-measures comprises actuating the automated valve to direct the fluid to the waste tank before contaminated fluid reaches the automated valve.

Additionally, or alternatively, automated valves can be used to sort milk by desired composition (rather than or in addition to contamination). Monitoring milk composition allows sorting milk by desired composition—for example high fat, high protein content, as in most countries a surplus in milk fats, proteins and/or lactose is subject to premium price, thus allowing the farm to maximize the profit of the business

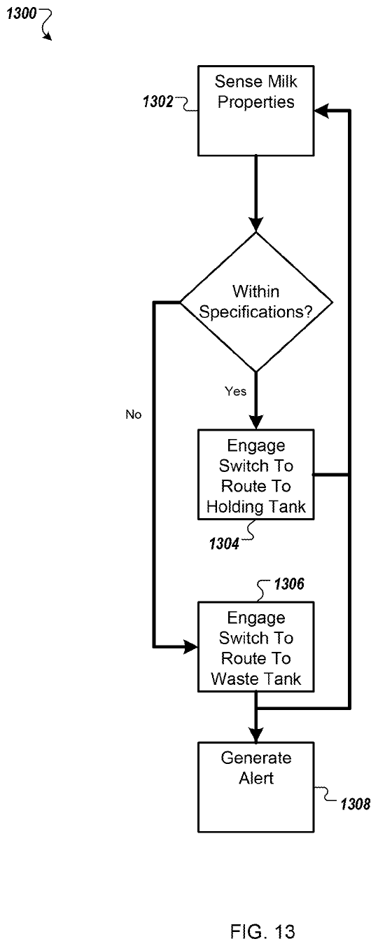

FIG. 13 shows a flowchart of an example process 1300 for diverting contaminated fluids away from a holding tank and into a waste tank. For example, the process 1300 may be used in actuating 714 a controllable device 108.

Milk properties are sensed 1302. For example, contamination levels can be determined, fat contact can be determined, or other appropriate parameters can be determined as previously described.

If the properties are within specification, a switch is engaged 1304 to route the milk to a holding tank. For example, the valve 108 may be put into a first position to route milk to the collection tank 110.

If the properties are out of specification (e.g., too much contamination, too little milk protein, improper temperature), the same switch is engaged 1306 to route the milk to a waste tank. For example, the valve 108 may be put into a second position to route the milk to the waste tank 112.

When the milk is out of specification, an alert can be generated 1308. The alert may include a physical event such as a light being illuminated by computer control, an auditory alarm played, etc. The alert may include a data message being sent over a network or stored in memory, such as a push-notification on an application, and email being sent, etc.

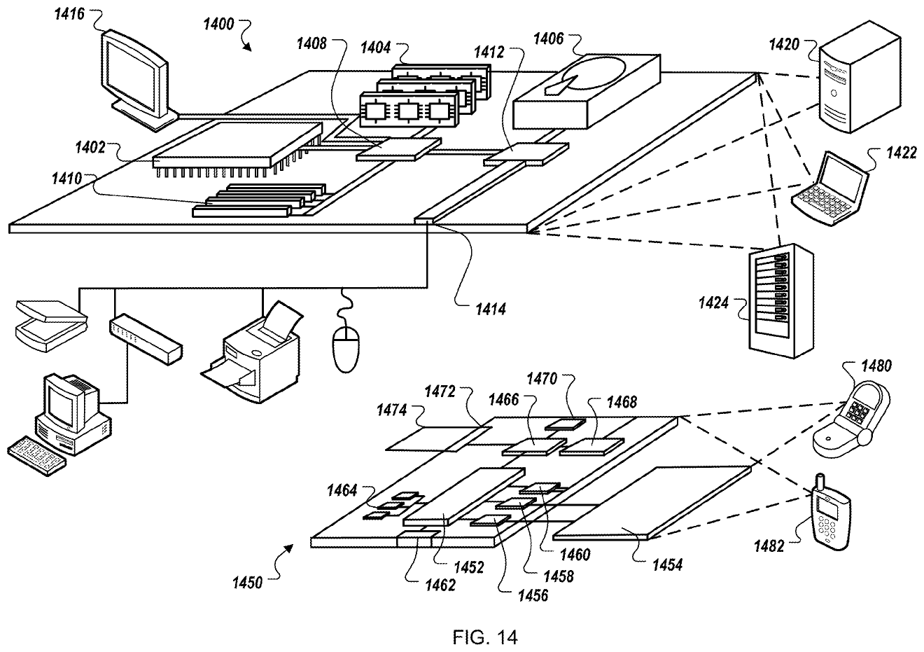

FIG. 14 shows an example of a computing device 1400 and an example of a mobile computing device that can be used to implement the techniques described here. The computing device 1400 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1400 includes a processor 1402, a memory 1404, a storage device 1406, a high-speed interface 1408 connecting to the memory 1404 and multiple high-speed expansion ports 1410, and a low-speed interface 1412 connecting to a low-speed expansion port 1414 and the storage device 1406. Each of the processor 1402, the memory 1404, the storage device 1406, the high-speed interface 1408, the high-speed expansion ports 1410, and the low-speed interface 1412, are interconnected using various busses, and can be mounted on a common motherboard or in other manners as appropriate. The processor 1402 can process instructions for execution within the computing device 1400, including instructions stored in the memory 1404 or on the storage device 1406 to display graphical information for a graphical user interface (GUI) on an external input/output device, such as a display 1416 coupled to the high-speed interface 1408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1404 stores information within the computing device 1400. In some implementations, the memory 1404 is a volatile memory unit or units. In some implementations, the memory 1404 is a non-volatile memory unit or units. The memory 1404 can also be another form of computer-readable medium, such as a magnetic or optical disk.

The storage device 1406 is capable of providing mass storage for the computing device 1400. In some implementations, the storage device 1406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer-or machine-readable medium, such as the memory 1404, the storage device 1406, or memory on the processor 1402.

The high-speed interface 1408 manages bandwidth-intensive operations for the computing device 1400, while the low-speed interface 1412 manages lower bandwidth-intensive operations. Such allocation of functions is exemplary only. In some implementations, the high-speed interface 1408 is coupled to the memory 1404, the display 1416 (e.g., through a graphics processor or accelerator), and to the high-speed expansion ports 1410, which can accept various expansion cards (not shown). In the implementation, the low-speed interface 1412 is coupled to the storage device 1406 and the low-speed expansion port 1414. The low-speed expansion port 1414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1400 can be implemented in a number of different forms, as shown in the figure. For example, it can be implemented as a standard server 1420, or multiple times in a group of such servers. In addition, it can be implemented in a personal computer such as a laptop computer 1422. It can also be implemented as part of a rack server system 1424. Alternatively, components from the computing device 1400 can be combined with other components in a mobile device (not shown), such as a mobile computing device 1450. Each of such devices can contain one or more of the computing devices 1400 and the mobile computing device 1450, and an entire system can be made up of multiple computing devices communicating with each other.

The mobile computing device 1450 includes a processor 1452, a memory 1464, and an input/output device such as a display 1454, a communication interface 1466, and a transceiver 1468, among other components. The mobile computing device 1450 can also be provided with a storage device, such as a micro-drive or other device, to provide additional storage. Each of the processor 1452, the memory 1464, the display 1454, the communication interface 1466, and the transceiver 1468, are interconnected using various buses, and several of the components can be mounted on a common motherboard or in other manners as appropriate.

The processor 1452 can execute instructions within the mobile computing device 1450, including instructions stored in the memory 1464. The processor 1452 can be implemented as a chipset of chips that include separate and multiple analog and digital processors. The processor 1452 can provide, for example, for coordination of the other components of the mobile computing device 1450, such as control of user interfaces, applications run by the mobile computing device 1450, and wireless communication by the mobile computing device 1450.

The processor 1452 can communicate with a user through a control interface 1458 and a display interface 1456 coupled to the display 1454. The display 1454 can be, for example, a TFT (Thin-Film-Transistor Liquid Crystal Display) display or an OLED (Organic Light Emitting Diode) display, or other appropriate display technology. The display interface 1456 can comprise appropriate circuitry for driving the display 1454 to present graphical and other information to a user. The control interface 1458 can receive commands from a user and convert them for submission to the processor 1452. In addition, an external interface 1462 can provide communication with the processor 1452, so as to enable near area communication of the mobile computing device 1450 with other devices. The external interface 1462 can provide, for example, for wired communication in some implementations, or for wireless communication in other implementations, and multiple interfaces can also be used.

The memory 1464 stores information within the mobile computing device 1450. The memory 1464 can be implemented as one or more of a computer-readable medium or media, a volatile memory unit or units, or a non-volatile memory unit or units. An expansion memory 1474 can also be provided and connected to the mobile computing device 1450 through an expansion interface 1472, which can include, for example, a SIMM (Single In-Line Memory Module) card interface. The expansion memory 1474 can provide extra storage space for the mobile computing device 1450, or can also store applications or other information for the mobile computing device 1450. Specifically, the expansion memory 1474 can include instructions to carry out or supplement the processes described above, and can include secure information also. Thus, for example, the expansion memory 1474 can be provided as a security module for the mobile computing device 1450, and can be programmed with instructions that permit secure use of the mobile computing device 1450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer-or machine-readable medium, such as the memory 1464, the expansion memory 1474, or memory on the processor 1452. In some implementations, the computer program product can be received in a propagated signal, for example, over the transceiver 1468 or the external interface 1462.

The mobile computing device 1450 can communicate wirelessly through the communication interface 1466, which can include digital signal processing circuitry where necessary. The communication interface 1466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1468 using a radio-frequency. In addition, short-range communication can occur, such as using a Bluetooth, WiFi, or other such transceiver (not shown). In addition, a GPS (Global Positioning System) receiver module 1470 can provide additional navigation-and location-related wireless data to the mobile computing device 1450, which can be used as appropriate by applications running on the mobile computing device 1450.

The mobile computing device 1450 can also communicate audibly using an audio codec 1460, which can receive spoken information from a user and convert it to usable digital information. The audio codec 1460 can likewise generate audible sound for a user, such as through a speaker, e.g., in a handset of the mobile computing device 1450. Such sound can include sound from voice telephone calls, can include recorded sound (e.g., voice messages, music files, etc.) and can also include sound generated by applications operating on the mobile computing device 1450.

The mobile computing device 1450 can be implemented in a number of different forms, as shown in the figure. For example, it can be implemented as a cellular telephone 1480. It can also be implemented as part of a smart-phone 1482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

1. A sensing system for sensing physical properties of a fluid, the system comprising:
a laser engine comprising configured to emit spectrally tunable laser radiation through the fluid as the fluid flows through the sensing system;
a laser detector configured to receive the laser radiation after the laser radiation has passed through the fluid and to generate corresponding laser-readings;
one or more processors;
computer memory storing computer-readable instructions that, when executed by the one or more processors, cause the one or more processors to perform operations comprising:
receiving, from the laser detector, laser-readings from the laser detector;
identifying, from the laser-readings, spectral-data reflective of physical properties of the fluid as the fluid flows through the sensing system; and
determining one or more fluid-measures for corresponding one or more constituents of the fluid using the spectral-data and reference-data defining one or more reference spectra for each possible constituent of the fluid.

2. The system of claim 1, wherein the system further comprises a housing containing the laser engine, the laser detector, and one or more processors, and the computer memory.

3. The system of claim 2, wherein the system further comprises a network interface, and wherein the operations further comprise transmitting, through the network interface, the one or more fluid-measures.

4. The system of claim 1, wherein the system further comprises:
a housing containing the laser engine and the laser detector; and
one or more computing devices comprising the one or more processors and the computer memory.

5. The system of claim 1, wherein the system further comprises a fluid channel through which the fluid flows between the laser engine and the laser detector, and wherein the laser engine and laser detector are fixedly held at least partly in the fluid channel such that a portion of the fluid flows between the laser engine and the laser detector.

6. The system of claim 5, wherein the system further comprises:
a contact sensor fixedly held at least partly in the fluid channel;
an optical emitter fixedly held at least partly in the fluid channel; and
a color sensor fixedly held at least partly in the fluid channel.

7. The system of claim 5, wherein the laser engine and laser detector are fixedly held apart at a distance of less than 10 mm.

8. The system of claim 5, wherein the laser engine is coupled to a photon-permeable sheath configured to prevent contact of the laser engine by the fluid.

9. The system of claim 1, wherein the system further comprises a contact sensor configured to:
contact the fluid as the fluid flows through the sensing system;
sense one or more contact-phenomenon of the fluid to create corresponding contact-readings; and
wherein determining one or more fluid-measures further comprises using the contact-readings.

10. The system of claim 1, wherein the system further comprises:
an optical emitter configured to emit a non-coherent light; and
a color sensor configured to receive the non-coherent light after the non-coherent light has passed through the fluid and to generate corresponding non-coherent-readings; and
wherein determining one or more fluid-measures further comprises using the non-coherent-readings.

11. The system of claim 1, wherein the system further comprises an automated valve configured to selectively direct a flow of the fluid to a plurality of output channels;
and wherein the operations comprise actuating the automated valve based on at least one of the fluid-measures.

12. The system of claim 11, wherein actuating the automated valve comprises engaging the automated valve to direct the fluid to a waste tank responsive to determining a fluid-measure for a contaminant is greater than a threshold value to direct the fluid to the waste tank before contaminated fluid reaches the automated valve.

13. (canceled)

14. The system of claim 1, wherein the fluid is raw milk harvested from livestock.

15. (canceled)

16. (canceled)

17. (canceled)

18. (canceled)

19. (canceled)

20. (canceled)

21. (canceled)

22. (canceled)

23. (canceled)

24. (canceled)

25. The system of claim 1, wherein the fluid-measures are each measures of constituents at a particular time, the operations further comprise:
aggregating the fluid-measures of a single milking session into an aggregate that reflects changes to the fluid-measure through the milking session.

26. The system of claim 1, wherein the operations further comprise:
storing the fluid-measures of a single milking session indexed by a unique identifier for the single milking session for an individual animal.

27. The system of claim 26, wherein the operations further comprise:
storing the fluid-measures of a single milking session with other fluid-measures of other milking sessions to generate historic data for a particular animal.

28. The system of claim 27, wherein the operations further comprise:
generating herd-wise metrics for a plurality of animals using historic data for each animal in a herd.

29. The system of claim 28, wherein the operations further comprise:
matching the herd-wise metrics with third party data to at least one of the group comprising 1) herd health data, 2) herd selection data, and 3) herd management data.

30. The system of claim 29, wherein generation of individual or herd-wise metrics includes the use of the third party data and historic data for each animal in a herd.

31. The system of claim 1, wherein physical properties of the fluid include fluid composition, electrical properties, temperature, flow, color, quantitate constituent concentration levels, absence or presence of one or more constituents.
